SEQUENCE LISTING (34 sequences)

SEQ ID NO: 1 - protein, 326 aa, Avian orthoreovirus
MAGLNPSQRREVVSLILSLTSNVNISHGDLTPIYERLTNLEASTELLHRSISDISTTVSNISANLQDMTHTLDDVTANLDGLRTTVTALQDSVSILSTNVTDLTNRSSAHAAILSSLQTTVDGNSTAISNLKSDISSNGLAITDLQDRVKSLESTASHGLSFSPPLSVADGVVSLDMDPYFCSQRVSLTSYSAEAQLMQFRWMARGTNGSSDTIDMTVNAHCHGRRTDYMMSSTGNLTVTSNVVLLTFDLSDITHIPSDLARLVPSAGFQAASFPVDVSFTRDSATHAYQAYGVYSSSRVFTITFPTGGDGTANIRSLTVRTGIDT

SEQ ID NO: 2 - protein, 205 aa, synthetic construct
  REGION 1..205: note = synthetic
DGNSTAISNLKSDISSNGLAITDLQDRVKSLESTASHGLSFSPPLSVADGVVSLDMDPYFCSQRVSLTSYSAEAQLMQFRWMARGTNGSSDTIDMTVNAHCHGRRTDYMMSSTGNLTVTSNVVLLTFDLSDITHIPSDLARLVPSAGFQAASFPVDVSFTRDSATHAYQAYGVYSSSRVFTITFPTGGDGTANIRSLTVRTGIDT

SEQ ID NO: 3 - protein, 205 aa, synthetic construct
  REGION 1..205: note = synthetic
QALLVEVSNLKSSVSSQGLTISNLERRVQALEGGSSTTLSFADPLKLEDGTVSLELDPYFCSVSRNLTSYSAGAQLMQFQWSVKGEDGAANSIDMDVNAHSHGPRTDYLMSTKQSLTVTTSPATLVFELDRITSLPSDLSRLIPCHGFQQATFPVDISFQRDGVSHTYQVYGKYSSSRVFTITFPTGGDGTANIRSLTVRTGIDT

SEQ ID NO: 4 - protein, 205 aa, synthetic construct
  REGION 1..205: note = synthetic
NGNSSAISNLRNDVSASGLNITDLQNRVKSLESDTSHGLSFSPPLSVADGVVSLDMDPYFCSQRVSLTSYSAEARLMQFQWVAKGTSGSSDTIDMTVNAHCHGRRTDYMMSSTGGLTVTSNAVSLTFDLSYITNMPSDLSRLIPSAGFQVASFPVDVSFTRESSTHTYQVYGAYSSARVFTITFPTGGNGTSNIRFLTVRTGIDT

SEQ ID NO: 5 - protein, 205 aa, synthetic construct
  REGION 1..205: note = synthetic
NANVTDIANLKGSVTTLSLTVTDLEKRLKVVESGSSSSLEFTSPLSLTDGVVSLNMDPYFCSDNHALTSYSSDAQLMQFQWLARGDDGSAGSVEMLVNAHCHGRRTDYMMSTTENLTVTGNSTSLVFSLDYITKPPSDMSRLVPRAGFQAASFPVDVSFTRDTTTHAYQVYGAYSSSRVFTITFPTGGDGTANIRSLTVRTGIDT

SEQ ID NO: 6 - protein, 267 aa, Avian orthoreovirus
GDLKSVYERLTSLEASTESLRQSVSGMSVTLSDLSADLQDTTRALDDVTVTLNNLSATITALQSSVTTLSATVDELTNTSSAHSGMLSSLQTIINGNSSAISNLRNDVSASGLNITDLQNRVKSLESDTSHGLSFSPPLSVADGVVSLDMDPYFCSQRVSLTSYSAEARLMQFQWVAKGTSGSSDTIGMTVNAHCHGRRTDYMMSSTGGLTVTSNAVSLTFDLSYITNMPSDLSRLIPSAGFQVASFPVDVSFTRESSTHTYQVYGA

SEQ ID NO: 7 - protein, 268 aa, Avian orthoreovirus
GDLKSVYERLNSLEASTESLRQSVSGMSVTLSDLSADLQDTTRALDDVTVTLNNLSATITALQSSVTTLSATVDELTNTSSAHSGMLSSLQTIINGNSSAISNLRNDVSASGFDISDLQNRVKSLESDTSHGLSFSPPLSVADGVVSLDMDPYFCSQRVSLTSYSAEARLMQFQWVAKGTSGSSDTIDMTVNAHCHGRRTDYMMSSTGGLTVTSNAVSLTFDLSYITNMPSDLSRLIPSAGFQVASFPVDVSFTRESSTHTYQVYGAY

SEQ ID NO: 8 - protein, 315 aa, Avian orthoreovirus
SSQRREVVSLILSLNSNVTINPGDLKSVYERLNSLEASTESLRQSVSGMSVTLSDLSADLQDTTRALDDVTVTLNNLSATITALQSSVTTLSATVDELTNTSSAHSGMLSSLQTIINGNSSAISNLRNDVSASGLNITDLQNRVKSLESDTSHGLSFSPPLSVADGVVSLDMDPYFCSQRVSLTSYSAEARLMQFQWVAKGTSGSSDTIDMTVNAHCHGRRTDYMMSSTGGLTVTSNAVSLTFDLSYITNMPSDLSRLIPSAGFQVASFPVDVSFTRESSTHTYQVYGAYSSARVFTITFPTGGNGTSNIRFLTV

SEQ ID NO: 9 - protein, 301 aa, Avian orthoreovirus
QRREVVSLILSLTSNATINPGDLTSIRERLTSLEASTESLSRSISSMSTTVSDLSADLQNVTRALDDVTANLNGIRVTITTLQDSVSTLSTTVTDLSRTSSAHSEALSSLRTIVDGNSTIIGNLKSDVASNGLAITDLQSRVKSLESASSHGLSFSPPLSVADGVVSLNLDPYFCSQRVSLTSYSAEALLMQFQWMARGTNGSADNIDMTVNAHCHGRRTDYIMSSTGGLTVTQNAVSLTFDLSYITRFPSDLSRLIPSAGFQAASFPVDVSFTRGSTTHTYQAYGVYSSSRIFTITFPTG

SEQ ID NO: 10 - protein, 316 aa, Avian orthoreovirus
QRREVVSLILSLNSNVTINPGDLKSVYERLTSLEASTESLRQSVSGMSVTLSDLSADLQDTTRALDDVTVTLNNLSATITALQSSVTTLSATVDELTNTSSAHSGMLSSLQTIINGNSSAISNLRNDVSASGLNITDLQNRVKSLESDTSHGLSFSPPLSVADGVVSLDMDPYFCSQRVSLTSYSAEARLMQFQWVAKGTSGSSDTIDMTVNAHCHGRRTDYMMSSTGGLTVTSNAVSLTFDLSYITNMPSDLSRLIPSAGFQVASFPVDVSFTRESSTHTYQVYGAYSSARVFTITFPTGGNGTSNIRFLTLRTG

SEQ ID NO: 11 - protein, 318 aa, Avian orthoreovirus
SQRREVVSLILSLTSNATINPGDLTSIRERLTSLEASTESLSRSISSMSTTVSDLSADLQNVTRALDDVTANLNGIRVTITTLQDSVSTLSTTVTDLSRTSSAHSESLSSLRTIVDGNSTIIGNLKSDVASNGLAITDLQSRVKPLESASSHGLSFSPPLSVADGVVSLNLDPYFCSQRVSLASYSAEALLMQFQWMARGTNGSADNIDMTVNAHCHGRRTDYIMSSTGGLTVTQNAVSLTFDLSYITRFPSDLSRLIPSAGFQAASFPVDVSFTRGSTTHTYQAYGVYSSSRIFTITFPTGGDGPANIRFLTVRTGI

SEQ ID NO: 12 - protein, 267 aa, Avian orthoreovirus
GDLMQIHERLSALELTTTSLNDSVNTALSKVTDLSGSLDNMAVSLAETEGELIKLSSAVQCLRTSLDHLLQNWRLCPHWYVTHGSSISDLQKEGQALSSEVNNLNLHVSSQGLTVSNLERRVQALEGGSSTTLSFADPLKFEDGTVSLELDPYFCSVSRNLTSYSAGAQLMQFQWSVKGEDGAANSIDMDVNAHSHGPRTDYLMSTKQSLTVTSLLATLVFELGRITSLPSDLSRLIPCHGFQQATFPVDISFQRDGVSHTYQVYGK

SEQ ID NO: 13 - protein, 305 aa, Avian orthoreovirus
LILSLTSSVTISPGDLMQIHERLSALELTTTSLNDSVNTALSKVTDLSGSLDNMAVSLAETKVELSSLSSAVQGLRTSLESSASELASLSSLVRNHGSAISDLQKEGQVLLVEVSNLKSSVSSQGLTISNLERRVQALEGGSSTTLSFADPLKLEDGTVSLELDPYFCSVSRNLTSYSAGAQLMQFQWSVKGEDGAANSIDMDVNAHSHGPRTDYLMSTKQSLTVTTSPATLVFELDRITSLPSDLSRLIPCHGFQQATFPVDISFQRDGVSHTYQVYGKYVSSRVFKITFSPGSSGPAVVQFLT

SEQ ID NO: 14 - protein, 312 aa, Avian orthoreovirus
QRREVVGLILSLTSSVTISPGDLTQIHERLSALELTTTSLNDSVNTALSKVTDLSGSLDNMAVSLAETKVELSSLSSAVQGLRTSLESSASELASLSSLVRNHGSAISDLQKEGQVLLVEVSNLKSSVSSQGLTISNLERRVQALEGGSSTTLSFADPLKLEDGTVSLELDPYFCSVSRNLTSYSAGAQLMQFQWSVKGEDGAANSIDMDVNAHSHGPRTDYLMSTKQSLTVTTSPATLVFELDRITSLPSDLSRLIPCHGFQQATFPVDISFQRDGVSHTYQVYGKYVSSRVFKITFSPGSSGPAVVQFLT

SEQ ID NO: 15 - protein, 260 aa, Avian orthoreovirus
VVGLILSLTSSVTISPGDLMQIHERLSALESAITSLNESVDTALSKLVDLSGSLDSMAVSLAETKVELHSLSSDVKGLRTSLDSSASELASLSSLVHDHGSSISDLQKGSRVLSVEVDNLKSSVSSQGLMISSLESRVQALEGGPGTNLSFADPLKLEDGTVSLELDPYFCSVSRNLTSYSADAQLMQFQWSAKGEDGAANSIDMDVNAHSHGSRTDYLMSTKQSLTVTTSPATLVFELGRITALPSDLSRLIPCHGFQQ

SEQ ID NO: 16 - protein, 254 aa, Avian orthoreovirus
QRREVVGLILSLTSSVTISPGDLMRIHERLSALELTTTSLNDSVNTALSKVTDLSGSLDNMAVSLAETKVELSSLSSAVQGLRTSLESSASELASLSSLVRNHGSAISDLQKEGQALLVEVSNLKSSVSSQGLTISNLERRVQALEGGSSTTLSFADPLKLEDGTVSLELDPYFCSVSRNLTSYSAGAQLMQFQWSVKGEDGAANSIDMDVNAHSHGPRTDYLMSTKQSLTVTTSPATLVFELDRITSLPSDLS

SEQ ID NO: 17 - protein, 267 aa, Avian orthoreovirus
GDLTTIYERLLKLDSSVESLTISVGDLSRKLSELEVDLQNIDSSMHQLTSSLTTLSEEVRQLRSAVGDNTDSISSLSTMVSDHQQLLADLRTSLNANVTDITNLKGGVTTLSLTVTDLEKRLKVVESGSSSSLEFTSPLSLTDGVVSLNMDPYFCSDNHALTSYSSDAQLMQFQWLARGDDGSAGSVEMLVNAHCHGRRTDYMMSTTENLTVTGNSTSLVFSLDYITKPPSDMSRLVPRAGFQAASFPVDVSFTRDTTTHAYQVYGA

SEQ ID NO: 18 - protein, 267 aa, Avian orthoreovirus
GDLTTIYERLLKLDSSVESLTISVGDLSRKLSELEVDLQNIDSSMHQLTSSLTTLSEEVRQLRSAVGDNTDSISSLSTMVSDHQQLLADLQTSLNANVTDITNLKGSVTTLSLTVTDLEKRLKVVESGSSSSLEFTSPLSLTDGVVSLNMDPYFCSDNHALTSYSAGAQLMQFQWLARGDDGSAGSVEMLVNAHCHGRRTDYMMSTTENLTVTGNSTSLVFSLDYITKPPSDMSRLVPRAGFQAASFPVDVSFTRDTTTHAYQVYGA

SEQ ID NO: 19 - protein, 267 aa, Avian orthoreovirus
GDLTTIYERLLKLDSSVESLTISAGDLSRRFSELEVDLQNVNSSMHQLTSSLTTLSEEARQLRSAVSDNTASISSLSTMVSGHQQLLADLQTSLNANVTDIANLKGSVTTLSLTVTDLEKRLKVVESGSSSSLEFTSPLSLTDGVVSLNMDPYFCSDNHALTSYSSDAQLMQFQWLARGDDGSAGSVEMLVNAHCHGRRTDYMMSTTENLTVTGNSTSLVFSLDYITKPPSDMSRLVPRAGFQAASFPVDVSFTRDTTTHAYQVYGA

SEQ ID NO: 20 - protein, 267 aa, Avian orthoreovirus
GDLTTIYERLLKLDSSVESLTISAGDLSRRFSELEVDLQNVNSSMHQLTSSLTTLSEEARQLRSAVSDNTASISSLSTMVSGHQQLLADLQTSLNANVTDIANLKGSVTTLSLTVTDLEKRLKVVESGSSSSLEFTSPLSLTDGVVSLNMDPYFCSDNHALTSYSSDAQLMRFQWLARGDDGSAGSVEMLVNAHCHGRRTDYMMSTTENLTVTGNSTSLVFSLDYITKPPSDMSRLVPRAGFQAASFPVDVSFTRDTTTHAYQVYGA

SEQ ID NO: 21 - protein, 267 aa, Avian orthoreovirus
GDLASIHDRLLKLDSTVESLTASVGDLSQKCIDLESNFQSVESSLGQVTSSLTDLSTEIRQLRLSLNDITSSMASLSTTVSDHDNSLSTLRVSVQTNVTDIANLKSSVNTLSLTVADLEKRLGVVESGSSSSLRFSSPLSLNNGVVSLDMDPYFCSDNHALTSYSSDAQLMQFQWLARGDDGSSGSVEMLVNAHCHGRRTDYMMSTTENLTVTGNSTSLVFSLDYITRPPSDMSRLIPRAGFQAASFPVDVSFTRDTTTHAYQVYGA

SEQ ID NO: 22 - protein, 296 aa, Avian orthoreovirus
  SITE 7: note = misc_feature - Xaa can be any naturally occurring amino acid
YCSLTSXREYKLWEHLASIHDRLLKLDSTVESLTASVGDLSQKCIDLESNFQSVESSLGQVTSSLTDLSTEIRQLRLSLNDIASSMASLSTTVSDHDNSLSTLRVSVQTNVTDIANLKSSVSTLSLTVADLEKRLGVVESGSSSSLRFSSPLSLNNGVVSLDMDPYFCSDNHSLTSYSSDAQLMQFQWLARGDDGSSGSVEMLVNAHCHGRRTDYMMSTTENLTVTGNSTSLVFSLDYITRPPSDMSRLIPRAGFQAASFPVDVSFTRDTTTHAYQVYGAYTTPRLSRLRFLTGGT

SEQ ID NO: 23 - protein, 267 aa, Avian orthoreovirus
GDLTPIYERLTNLEASTELLHRSISDISTTVSNISASLQDMTHTLDDVTANLDGLRTTVTALQDSVSILSTNVTDLTNTSSAHAATLSSLQTTVDGNSTAISNLKSDVSSNGLAITDLQDRVKSLESTASHGLSFSPPLSVADGVVSLDMDPYFCSQRVSLTSYPAEAQLMQFRWMARGTNGSSDTIDMTVNAHCHGRRTDYMMSSTGNLTVTSNVVLLTFDLSDITHIPSDLARLVPSAGFQAASFPVDVSFTRDSATHAYQAYGV

SEQ ID NO: 24 - protein, 267 aa, Avian orthoreovirus; identical to SEQ ID NO: 18

SEQ ID NO: 25 - protein, 267 aa, Avian orthoreovirus; identical to SEQ ID NO: 19

SEQ ID NO: 26 - protein, 321 aa, Avian orthoreovirus
LVISSQRREVVSLILSLNSNVTINPGDLKSVYERLNSLEASTESLRQSVSGMSVTLSDLSADLQDTTRALDDVTVTLNNLSATITALQSSVTTLSATVDELTNTSSAHSGMLSSLQTIINGNSSAISNLRNDVSASGLNITDLQNRVKSLESDTSHGLSFSPPLSVADGVVSLDMDPYFCSQRVSLTSYSAEARLMQFQWVAKGTSGSSDTIDMTVNAHCHGRRTDYMMSSTGGLTVTSNAVSLTFDLSYITNMPSDLSRLIPSAGFQVASFPVDVSFTRESSTHTYQVYGAYSSARVFTITFPTGGNGTSNIRFLTVRTG

SEQ ID NO: 27 - protein, 294 aa, Avian orthoreovirus
WIDTVVDFERDYKSWRFDANPERLSALELTTTSLNDSVNTALSKVTDLSGSLDNMAVSLAETKVELSSLSSVVQGLRTSLESSASELASLSSLVRNHGSSISDLQKEGQALLVEVNNLKSSVSSQGLTISNLERRVQALEGGSSTTLSFADPLKLEDGTVSLELDPYFCSVSRNLTSYSASAQLMQFQWSVKGEDGAANSIDMDVNAHSHGPRTDYLMSTKQSLTVTTSPATLVFELDRITSLPSDLSRLIPCHGFQQATFPVDISFQRDGVSHTYQVYGKYASSRVFKITFSP

SEQ ID NO: 28 - protein, 126 aa, Avian orthoreovirus
NIKPGDLKSVYERLTSLEASTESLRQSVSGMSVTLSDLSADLQDTTRALDDVTVTLNNLSATITALQSSVTTLSATVDELTNTSSAHSGMLSSLQTIINGNSSAISNLRNDVSASGLNITDLQDRV

SEQ ID NO: 29 - protein, 116 aa, Avian orthoreovirus
DAQLMQFQWLARGDDGSSGSVEMLVNAHCHGRRTDYMMSTTENLTVTGNSTSLVFSLDYITRPPSDMSRLIPRAGFQAASFPVDVSFTRDTTTHAYQVYGAYTTPRLFKITFPTGR

SEQ ID NO: 30 - protein, 267 aa, Avian orthoreovirus
GDLIPLYERLSAVEKMCATVNDSLGRLTSLVSEMSARIDSLADTLQENAAGLDQRSTVTTLSVSLFDDLSGRVATLPLMVSNQESQLPSSLPSVNALSTTCSNLQRDVSSTALTVTSLGQRVEALESGAGSDLTFMAPLKVDGKSVSLDMDPYFCSERTNLTSYSANAQLLQFQWLVRSEGESSDSIDMNVVAHCHGRRTDYLMSTHDSLTVVGNSVTLIFNLDFITLKASDYARLVPCHGFQQATFPVDISFTKGTATQSYQVYGA

SEQ ID NO: 31 - protein, 267 aa, Avian orthoreovirus; identical to SEQ ID NO: 12

SEQ ID NO: 32 - protein, 162 aa, Avian orthoreovirus
QSQRREVGGLILSLTSSVTISPGDLMRIHERLSALELTTTSLNDSVNTALSKVTDLSGSLDNMAVSLAETKVELSSLSSAVQGLRTSLESSASELASLSSLVRNHGSAISDLQKEGQALLVEVSNLKSSVSSQGLTISNLERQVQALEGGSSTTLSFADPLK

SEQ ID NO: 33 - protein, 229 aa, Avian orthoreovirus
ERLSALESAITSLNESVDTALSKLVDLSGSLDSMAVSLAETKVELHSLSSDVKGLRTSLDSSASELASLSSLVHDHGSSISDLQKGSRVLSVEVDNLKSSVSSQGLMISSLESRVQALEGGPGTNLSFADPLKLEDGTVSLELDPYFCSVRRNLTSYSADAQLMQFQWSAKGEDGAANSIDMDVNAHSHGSRTDYLMSTKQSLTVTTSPATLVFELDRITALPSDFSSL

SEQ ID NO: 34 - protein, 302 aa, Avian orthoreovirus
ISPGDLVPVYDRLSAVEAAYATLNASVGQLTSTMSDLSIRLDDLANAIQSTSTDLRDVQSRVSALQTSVDGISSDVATLSQSLSTHGSQLSALSSSVSTLSTDMSNIQRDVASSALNVADLQRRVTALESGAGSSLTFLAPLRADGGSVSLDMDPYFCSERSNLTSYSASAQLLQFQWFVRSEGGSSDSIDMSVVAHCHGRRTDYLMSSHDSLTVTGNSVSLVFNLDYITTSGVDYARLIPCHGFQQATFPVDISFTKNDATHTYQVYGAFDGPRIFKVTFSPGETSTTNIRFITVRTGIEI

(19) United States
(12) Patent Application Publication
(10) Pub. No.: US 2024/0050556 A1
(43) Pub. Date: Feb. 15, 2024

(54) OPTIMIZED POLYPEPTIDE FOR A SUBUNIT VACCINE AGAINST AVIAN REOVIRUS

(71) Applicant: GAVISH-GALILEE BIO APPLICATIONS, LTD, Kiryat Shmona (IL)

(72) Inventors: Jacob PITCOVSKI, Korazim (IL); Dana Goldenberg, Lower Galilee (IL)

(73) Assignee: GAVISH-GALILEE BIO APPLICATIONS, LTD, Kiryat Shmona (IL)

(21) Appl. No.: 18/357,544

(22) Filed: Jul. 24, 2023

Related U.S. Application Data
(63) Continuation of application No. 18/067,988, filed on Dec. 19, 2022, now abandoned.
Application No. 18/067,988 is a continuation of application No. 16/068,548, filed on Jul. 6, 2018, now Pat. No. 11,541,111.
(60) Provisional application No. 62/275,838, filed on Jan. 7, 2016.

(51) Int. Cl. A61K 39/15; A61P 31/12; C07K 14/005; C12N 15/63; A61K 39/12

(52) U.S. Cl. CPC A61K 39/15; A61P 31/12; C07K 14/005; C12N 15/63; A61K 39/12; A61K 2039/552

(57) ABSTRACT

An isolated polypeptide comprising an amino acid sequence corresponding to the amino acid residues forming a full or partial α-helical domain, the hinge domain, the β-triple spiral domain and a full or partial globular head domain of an avian reovirus sigma C protein, and lacking the amino acid sequence that is N-terminal to said α-helical domain is provided. Furthermore, a vaccine comprising, or a viral vector expressing, at least one of the isolated polypeptides of the present invention is provided.

### SEQUENCE LISTING

A sequence listing in electronic (XML file) format is filed with this application and incorporated herein by reference. The name of the ASCII text file is “GAVISH-067-US2.xml”; the file was created on Jul. 24, 2023; the size of the file is 40,768 bytes

### FIELD OF THE INVENTION

The present invention relates in general to vaccines against avian reovirus.

### BACKGROUND OF THE INVENTION

Avian reovirus (ARV) is a member of the Orthoreovirus genus in the family Reoviridae. It is associated with a number of diseases, the most prominent being viral arthritis syndrome (tenosynovitis) which is characterized by swelling of the hock joints and lesions in the gastrocnemius tendons, and causes considerable economic loss to the poultry industry. Susceptibility to ARV occurs mostly in young (1-2 weeks of age) chickens. The control of viral tenosynovitis in broiler chicks is conferred by antibodies that are transferred to the progeny following vaccination of maternal flocks. The available live-attenuated and inactivated vaccines for ARV are based on the s1133 strain (van der Heide et al. 1983), as well as isolated strains belonging to a single serotype (Goldenberg et al. 2010). However, those vaccines are not effective against the diverse ARVs found in the field (Jones 2000; Goldenberg et al. 2010; Lublin et al. 2011).

Sequencing of the sigma C (SC) protein of ARV isolates for genetic characterization enabled their division into genotypes (Kant et al. 2003; Lublin et al. 2011; Goldenberg et al. 2010; Troxler et al. 2013). Vaccination based on a mixture of the four representatives of ARV genotypes conferred protection against all tested viruses from the four genotypes [Lublin et al. 2011; WO 2009/093251]. The outer capsid cell attachment protein SC of ARV, encoded by the 51 gene, is a relatively small protein of 326 amino acids (Benavente & Martinez-Costas 2007), a homotrimer with a tertiary structure consisting of two domains: the “head”, which is located at the C-terminal end of the protein, and the “shaft”, at the N terminus. The crystal structure of the C-terminal domain and of residues 117-326 has been resolved (Guardado Calvo et al. 2005; Guardado-Calvo et al. 2009). SC elicits reovirus-specific neutralizing antibodies (Shapouri et al. 1996; Grande et al. 1997), making it a suitable candidate for a recombinant subunit vaccine.

Indeed, efficient recombinant vaccines have been developed in the past for a number of viruses, including vaccines for hepatitis B (McAleer et al. 1984) and for papillomavirus (Valentino & Poronsky 2015) for humans, as well as infectious bursal disease (IBD) (Pitcovski et al. 2003) and egg drop syndrome (Fingerut et al. 2003) for chickens and hemorrhagic enteritis virus for turkeys (Pitcovski et al. 2005). SC has been expressed in various expression systems, including bacteria, baculovirus, yeast, plants and mammalian cells. Recombinant SC proteins have been used for diagnostics to distinguish between strains. Anti-SC antibodies have been shown to neutralize the virus in cell lines [28,31]. In a previous study, SC expressed in bacteria showed only weak immunogenicity (Goldenberg et al. 2011; Vasserman et al. 2004). There remains therefore a great need for an efficient vaccine against ARV.

### SUMMARY OF INVENTION

In one aspect, the present invention is directed to an isolated polypeptide comprising, or essentially consisting of, an amino acid sequence corresponding to the amino acid residues forming a full or partial α-helical domain, the hinge domain, the β-triple spiral domain and a full or partial globular head domain of an avian reovirus sigma C protein, and lacking the amino acid sequence that is N-terminal to said α-helical domain.

In an additional aspect, the present invention provides a nucleic acid molecule comprising a nucleic acid sequence encoding at least one of the isolated polypeptides defined herein.

In another aspect, the present invention provides an expression vector comprising a control element, such as a promoter, operably linked to said nucleic acid molecule defined herein, wherein said expression vector is designed to replicate and express relevant genes in for example a bacterial, yeast or insect cell.

In a further aspect, the present invention provides a vaccine comprising at least one of the isolated polypeptides defined herein.

In still a further aspect, the present invention provides a vaccine comprising a mammalian expression vector, such as pcDNA3, comprising a control element, such as a promoter, operably linked to the nucleic acid molecule defined herein.

In yet an additional aspect, the present invention provides a viral vector, such as a recombinant Marek's disease (MD) virus, comprising a control element, such as a promoter, operably linked to the nucleic acid molecule defined herein.

In yet another aspect, the present invention is directed to any one of the vaccines or viral vectors defined herein, for use in vaccination of an avian species or for use in inducing an avian immune response conferring protection against avian reovirus.

In yet an additional aspect, the present invention is directed to a method for vaccinating an avian species against avian reovirus or for inducing an avian immune response conferring protection against avian reovirus, which comprises administering any one of the vaccines or viral vectors defined herein to a bird.

### BRIEF DESCRIPTION OF DRAWINGS

FIGS. 1A-B show efficient expression of Sigma C of ARV isolates; His-tagged residues 122-326 was expressed in E. coli BL21. Coomassie Brilliant Blue staining (A) show robust expression of proteins of the expected molecular size. Their identity was confirmed using mouse anti-His monoclonal antibody via western blot (B). Lane 1: soluble SC1133, Lane 2: soluble SC528, Lane 3: soluble SC5223, Lane 4: soluble SC5215, Lane 5: insoluble SC1133, Lane 6: insoluble SC528, Lane 7: insoluble SC5223, Lane 8: insoluble SC5215, Lane 9: negative control (Sigma C residues 1-326 without his tag, produced in E. coli). Lane 10: molecular weight marker. Arrow indicates sigma C protein.

FIG. 2 depicts a bar graph showing antibody titers following vaccination with recombinant proteins SC1-326, SC122-326 and SC192-326, all of which are sequences of ARV s1133. Controls: N.C—negative control (birds vaccinated with PBS in adjuvant), P.C—positive control (injected with ARV s1133). *** Significantly different at P<0.001.

FIG. 3 depicts a bar graph showing virus detection by antibodies raised following protein injection. The relative level of anti-ARV antibody following injection with SC1-326, SC122-326 and SC192-326 is presented as the ratio between the ODs of the tested sample (S) and anti-ARV 1133 antibodies, provided in the KIT, that serve as positive control (P) (S/P). A value greater than 0.2 is considered positive (recognizing the virus). N.C—negative control (birds vaccinated with PBS in adjuvant). *** Significantly different at P<0.001.

FIG. 4 depicts a bar graph showing proliferation of lymphocytes derived from spleens of birds vaccinated with PBS in adjuvant as negative control (N.C.), ARV vaccine strain s1133 as positive control (P.C.), SC1-326 or SC122-326, following stimulation with ARV. Cells were treated with concanavalin A (ConA) or lipopolysaccharide (LPS) as controls for nonspecific antigen (Ag) cell proliferation, ARV for specific Ag proliferation, or the medium itself as a negative control (N.C.). The measured absorbance is proportional to the number of viable cells. Results are presented as stimulation index (SI) representing the ratio between stimulated and non-stimulated cells. ***,** Significantly different at P<0.001 and 0.01, respectively.

FIG. 5 depicts a line graph showing the effect of primary vaccination with SC122-326 on the secondary response to ARV. Results are expressed as dilution end-points, tested by ELISA (optical density (OD) measured at 450nm). Negative control—vaccinated with PBS and adjuvant. Circles—Primary immune response to ARV vaccine strain s1133; Squares—Immune response to ARV after primary vaccination with SC122-326; Upright triangles—Immune response to ARV after primary vaccination with ARV; -Inverted triangles—Negative control. ***Significantly different at P<0.001.

FIG. 6 depicts a model of a partial structure (amino acids 117-326) of sigma C (SC) protein of avian reovirus strain s1133. Numbers represent amino acid positions in the protein structure. Adopted from Guardado-Calvo et al. [18].

### DETAILED DESCRIPTION OF THE INVENTION

Avian reovirus (ARV) mutates relatively fast and many variants exist in the field worldwide. Vaccines that protect against one genotype are inefficient against others (Lu et al. 2015; Vasserman et al. 2004; Goldenberg et al. 2010; Shapouri et al. 1995). Production of attenuated vaccine is a long process, and in the case of highly mutated viruses inefficient. Adaptation of vaccines to alterations in the field virus is by the use of inactivated vaccines that are produced by propagation of the virulent isolates in cell cultures or embryonated eggs and its subsequent inactivation. In the era of molecular biology, subunit vaccines may be produced by genetic engineering. In this method, only the relevant updated protein for induction of neutralizing antibodies is produced in an expression system. As shown previously [26,27], sigma C protein (SC) in its full 326-amino-acid form can be expressed in a bacterial expression system. However, although antibodies against s1133, a viral strain of ARV, do identify recombinant SC, a low titer of antibodies was obtained in response to immunization with this protein.

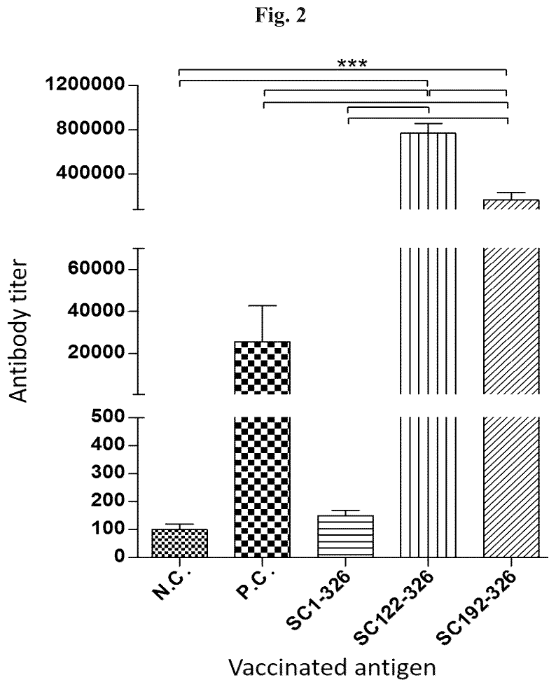

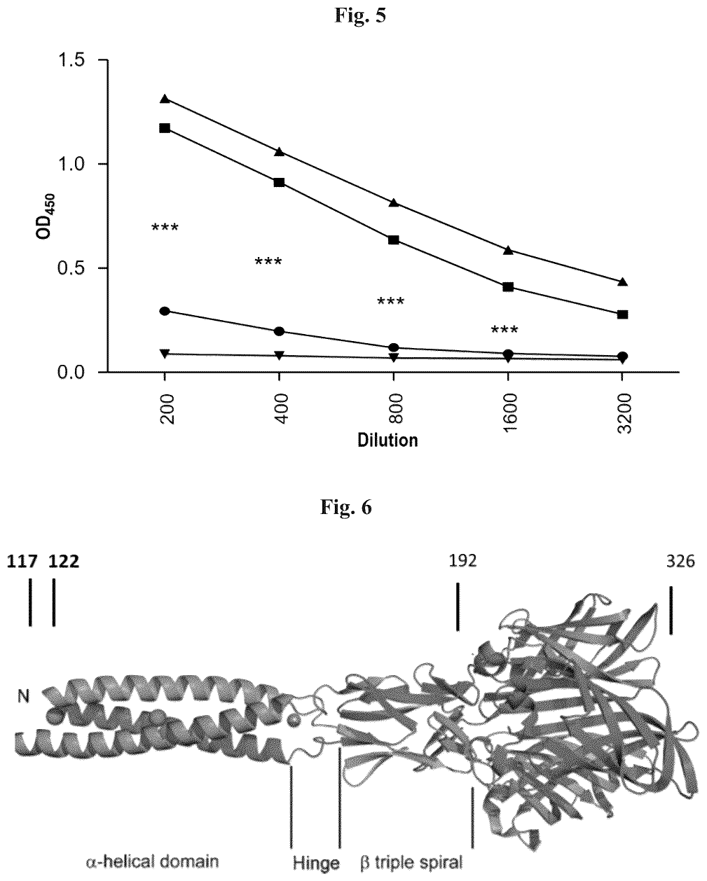

Three different fragments of the protein—residues 1-326, 122-326, and 192-326 (FIG. 5)—were identified and tested in accordance with the present invention as candidates for subunit vaccine that will elicit the highest levels of anti-ARV neutralizing antibodies. The present invention is based on the finding that while SC1-326 induces low titer of antibodies, the other two fragments induced an immune response with high antibody titers, the most prominent one being SC122-326 (FIG. 2). It was further found that following stimulating of splenocytes isolated from birds vaccinated with different SC polypeptides, only cells from birds that were exposed to SC122-326 polypeptide proliferated to the same extent as the positive control, which was vaccinated with the whole virus (FIG. 4).

As SC122-326 yielded the highest levels of antibodies, it was further analyzed. The ability to get a secondary immune response against ARV following priming with SC122-326 indicated that the same B and T lymphocytes are induced by SC122-326 and the whole virus (FIG. 5). This enables vaccinating at a young age with SC122-326 and overcomes the disruption of maternal antibodies in eliciting a full response.

The efficacy of SC122-326 as a vaccine against ARV was further tested by viral neutralization test. Antibodies produced against the SC protein showed successful neutralization in cell systems [28,45]. However, neutralization tests are more accurate at predicting viral neutralization in adult birds. It has thus been found in accordance with the present invention that antibodies produced following injection of SC122-326 protein are protective, eliminating infection of bird embryos by the virulent strain to the same extent as antibodies produced in response to the whole virus (Table 5). It is noteworthy that antibodies produced following injection of the whole SC protein (SC1-326) are not protective. It is thus self-evident that the isolated polypeptide SC122-326 has markedly different characteristics than the counterpart within the context of the whole protein.

In view of the above, in one aspect, the present invention is directed to an isolated polypeptide comprising, or essentially consisting of, an amino acid sequence corresponding to the amino acid residues forming a full or partial α-helical domain, the hinge domain, the β-triple spiral domain and a full or partial globular head domain of an avian reovirus sigma C protein as depicted in FIG. 6, and lacking the amino acid sequence that is N-terminal to said α-helical domain.

The term “isolated polypeptide” refers to the fact that the amino acid sequence is a fragment of the full-length sigma c protein, which is expressed outside its natural context within the full-length sigma c protein, and is used interchangeably herein with the terms “recombinant polypeptide”, “synthetic polypeptide” or “fragment of full-length sigma c protein”.

The different domains of the sigma C protein are well characterized as taught in Guardado-Calvo, 2009; When the overall structure is contemplated, a clear division between shaft (amino acid residues 117-191) and globular head domains (amino acid residues 196-326) is observed. The shaft domain can be further subdivided into an α-helical triple coiled-coil (amino acid residues 117-154), a linker region (amino acid residues 155-159) and two repeats of a triple β-spiral (amino acid residues 160-191).

In certain embodiments, the isolated polypeptide comprises, or essentially consists of, an amino acid sequence corresponding to amino acid residues 70-326, 117-326, or particularly 122-326, of the sigma C protein of the ARV strain S1133 as set forth in SEQ ID NO: 1, referred to herein as “the internal protein sequence of S1133” or “the internal amino acid sequence”. In particular, the internal amino acid sequence has at least 40%, at least 50%, at least 60%, at least 70%, at least 80%, at least 85%, at least 90%, or at least 95, 96, 97, 98, or 99% identity to the amino acid sequence corresponding to amino acid residues 70-326, 117-326, or 122-326 of the sigma C protein of the ARV strain S1133 as set forth in SEQ ID NO: 1.

In further particular embodiments, any one of the isolated polypeptides defined above blocks or reduces the binding of infectious avian reovirus to the native receptor of sigma C protein of an avian reovirus. Methods for measuring binding of ligands to proteins are well known in the art, for example, by surface plasmon resonance (SPR) as described in Barton Erik S, J.Craig Forres, Jodi L Connolly, James D Chappell, Yuan Liu, Frederick J Schnell, Asma Nusrat, Charles A Parkos, Terence S Dermody. Junction Adhesion Molecule Is a Receptor for Reovirus. Cell, Vol. 104, 441-451, Feb. 9, 2001.

In even more particular embodiments, any one of the isolated polypeptides defined above, when administered to a bird, optionally in combination with an adjuvant, induces a protective immune response against an infectious avian reovirus. In certain embodiments, when any one of the isolated polypeptides defined above is administered to a bird, it induces the production of significantly higher systemic levels of neutralizing anti-sigma C protein antibody as compared with the systemic levels of said antibody obtained after administration to a bird of full length sigma C protein of the ARV strain S1133. Methods for measuring induction of protective immune response in birds are well known in the art. For example, as taught in the Examples below, the antibody titer can easily be measured in the serum of vaccinated birds by ELISA test and the ability of the sera to neutralize virulent reovirus can easily be done by monitoring inhibition of embryonic mortality after inoculation with a live virus.

In certain embodiments, any one of the isolated polypeptides defined above comprises an amino acid sequence having at least 40%, at least 50%, at least 60%, at least 70%, at least 80%, at least 85%, at least 90%, or at least 95, 96, 97, 98, or 99% identity to the internal amino acid sequence of a sigma C protein selected from the group disclosed in Table 1, said internal amino acid sequence corresponding to amino acid residues 70-326, 117-326 or 122-326 of the sigma C protein of the ARV strain S1133 as set forth in SEQ ID NO: 1.

In certain embodiments, any one of the isolated polypeptides defined above comprises an amino acid sequence corresponding to an internal amino acid sequence within the amino acid sequence of a sigma C protein selected from the group disclosed in Table 1, said internal amino acid sequence corresponding to amino acid residues 70-326, 117-326 or 122-326 of the sigma C protein of the ARV strain S1133 as set forth in SEQ ID NO: 1.

In particular embodiments, the isolated polypeptide comprises, or essentially consists of, an amino acid sequence selected from the group consisting of SEQ ID NO: 2, SEQ ID NO: 3, SEQ ID NO: 4 and SEQ ID NO: 5, such as SEQ ID NO: 2 (i.e. internal protein sequence of S1133, ISR5223, ISR5215 and ISR528, respectively).

TABLE 1
Database accession numbers for representative
full length avian reovirus sigma c proteins.

| Isolate | Accession numbers¹ | SIN² | Isolate | Accession numbers | SIN² |
|---|---|---|---|---|---|
| S1133 | AAK18188.1 | 1 | ISR529 | FJ793528 | 20 |
| ISR521 | FJ793522 | 6 | ISR5211 | FJ793524 | 21 |
| ISR526 | FJ793537 | 7 | ISR5213 | FJ793533 | 22 |
| ISR5215 | FJ793531 | 8 | 59103 | AAQ01229.1 | 23 |
| ISR5220 | FJ793532 | 9 | ISR5212 | FJ793526 | 24 |
| ISR5225 | FJ793546 | 10 | ISR5210 | FJ793527 | 25 |
| ISR5226 | FJ793547 | 11 | ISR5216 | FJ793530 | 26 |
| ISR522 | FJ793540 | 12 | ISR5234 | FJ793534 | 27 |
| ISR5217 | FJ793535 | 13 | ISR5231 | FJ793536 | 28 |
| ISR5221 | FJ793542 | 14 | ISR5229 | FJ793538 | 29 |
| ISR5222 | FJ793543 | 15 | ISR525 | FJ793539 | 30 |
| ISR5223 | FJ793544 | 16 | ISR523 | FJ793541 | 31 |
| ISR524 | FJ793525 | 17 | ISR5224 | FJ793545 | 32 |
| ISR527 | FJ793529 | 18 | ISR5219 | FJ793548 | 33 |
| ISR528 | FJ793523 | 19 | ISR5233 | FJ793549 | 34 |

¹NCBI database at http://www.ncbi.nlm.nih.gov;
²SIN, SEQ ID NO:

In certain embodiments any one of the isolated polypeptides defined above further comprises a tag for identification and/or purification, such as a polyhistidine tag, (e.g. His₆), but it may also be a (His-Asn)₆ tag, a Flag tag, or any other tag that may facilitate the purification of the polypeptide. A sole tag or multiple copies of it can be used or different tags can be used in combination. The tag(s) can be fused to the C-terminus or the N-terminus of the polypeptide. For example, the isolated polypeptide having an internal amino acid sequence in a protein set forth in any one of SEQ ID Nos: 1 and 6-34, such as SEQ ID NO: 2, SEQ ID NO: 3, SEQ ID NO: 4 or SEQ ID NO: 5, may further comprises a tag for identification and/or purification, such as those described above.

For expression of the polypeptides described above, bacterial or eukaryotic cells, such as yeast or plant cells, may be transformed with the nucleic acid molecule and the expressed polypeptide may be purified using the purification tag inserted into the polypeptide and/or by other protein purification methods well known in the art. Preferably, the polypeptide is expressed in a bacterial cell, more preferably E. coli.

For use in immunogenic compositions/vaccines, any one of the polypeptides defined above may be combined with a carrier protein such as, but not limited to, E. coli heat labile enterotoxin (LT), bovine serum albumin and flagellin. The carrier protein may be fused to the N- or the C terminus of the polypeptide or it may flank the polypeptide on both ends. The carrier protein may be fused to the polypeptide by recombinant techniques, i.e. the polypeptide and the carrier protein are encoded by a single nucleic acid sequence and are expressed as a continuous polypeptide. Alternatively, the polypeptide and the carrier protein may be produced separately and then chemically attached to each other.

The preferred carrier protein for all aspects of the present invention is the LT protein. It confers the function of carrier and is also known as a powerful adjuvant in injection and oral administration that is capable of eliciting strong systemic IgG and local IgA responses as well as cytotoxic helper T-cell response. Since the reovirus is a mucosal antigen, it is preferential to stimulate the immune response of the mucosal system. IgA antibodies developed by oral vaccinations in mucosal tissues are the main deterrents against the challenge offered by mucosal antigens. The LT protein is a hexamer that consists of two subunits: the 27-kDa catalytic A domain (LTA) anchored in a ring of five identical 11.6-kDa B subunits (LTBs). The A1 fragment is toxic and catalyzes the transfer of an ADP-ribose from NAD to stimulatory α-subunits of G proteins (Gsa). To use LT as an immunostimulator in animals, its toxicity may be neutralized by mutations (Vasserman, Y. and Pitcovski, J., 2006). Either one of the A and B subunits may be used in conjunction with the polypeptide of the invention.

The polypeptide may also be attached to physiologically acceptable microbeads, which may carry enhancer molecules such as LT in addition to the polypeptide. Alternatively, the polypeptide may be bound indirectly to the carrier via antibodies or biotinylated polypeptides may be bound to the carrier via avidin or streptavidin.

Microparticles intended for use in the present invention preferably have a size in the range from 10 nm to 200 μm. The size chosen for a particular microparticle will depend on the active agent to be delivered, and the intended route of administration. For oral delivery, particles are conveniently in the size range 0.5 to 5.0 μm. For subcutaneous delivery, a suitable size is less than 100 μm. Microparticles for parenteral delivery conveniently have a size of less than 200 μm, preferably less than 150 μm.

In an additional aspect, the present invention provides a nucleic acid molecule comprising a nucleic acid sequence encoding at least one of the isolated polypeptides defined above.

In another aspect, the present invention provides an expression vector comprising a control element, such as a promoter, operably linked to the nucleic acid molecule defined above, wherein said expression vector is designed to replicate and express relevant genes in for example a bacterial, yeast or insect cell.

The invention further contemplates cells comprising and/or expressing any one of the expression vectors defined above. Examples of cells suitable for expressing the polypeptides of the present invention are bacterial cells, such as E.coli, yeast cells, insect cells, plant cells or mammalian cells.

In a further aspect, the present invention provides a vaccine comprising at least one of the isolated polypeptides defined above.

In still a further aspect, the present invention provides a vaccine comprising a mammalian expression vector, such as pcDNA3, comprising a control element, such as a promoter, operably linked to any one of the nucleic acid molecules defined above.

In yet an additional aspect, the present invention provides a viral vector, such as a recombinant Marek's disease (MD) virus, comprising a control element, such as a promoter, operably linked to any one of the nucleic acid molecules defined above.

WO 2009/093251, incorporated by reference as if fully disclosed herein, discloses that the highly variable and immunogenic avian reovirus sigma C protein amino acid sequence contains conserved and immunogenic domains that are common among all avian reovirus isolates identified in Israel and elsewhere. The identified conserved sequences are located between amino acid residue corresponding to amino acid residues 170 and 323 of the sigma C protein of the ARV strain S1133 as set forth in SEQ ID NO: 1.

The term “conserved domain” refers herein to a domain conserved among the virus variants; namely, the amino acid sequences of the domains are similar but not necessarily absolutely identical to each other.

WO 2009/093251 further discloses that the genetically variable avian reovirus strains, or isolates, infecting birds in Israel and elsewhere can be divided into four groups according to the variability in the amino acid sequence of the sigma C protein expressed by the isolates. One member of one of the groups represents only a fraction of the avian reovirus isolates in nature; however, one member of each of the four groups taken together represent many, if not most, variants existing in nature. The four groups of reovirus isolates (RI) are referred to herein as RI Groups Ito IV (Table 2).

The defining feature of each RI group is that the amino acid sequences of the different sigma c proteins of said RI group has at least 75% identity. The composition of the groups may vary slightly depending on the algorithm and software used to analyze the sequences. The important concept is that each group represents a fraction of the viral population and that single representatives taken from each one of the groups together represent the whole viral population.

Since the conserved immunogenic domains are found within the isolated polypeptide of the present invention, the concept disclosed in WO 2009/093251 can be used to produce an improved vaccine comprising isolated polypeptides of the present invention that represents the whole viral population.

It has been found in accordance with the present invention that the internal polypeptide of sigma c proteins of four different avian reovirus variants, each one of which corresponds to amino acid residues 122-326 of the sigma C protein of the ARV strain S1133 as set forth in SEQ ID NO: 1 can be effectively expressed in vitro.

In certain embodiments, the vaccine comprises at least two different recombinant polypeptides; or in the case of the vaccine comprising a mammalian expression vector or the viral vector, the nucleic acid molecule encodes at least two different recombinant polypeptides; and each one of said at least two different recombinant polypeptides is derived from a representative of one of two, three or four groups of different sigma c proteins, wherein the defining feature of each group is that the amino acid sequences of the different sigma c proteins of said group has at least 75% identity, optionally as defined in Table 2 and as taught in WO 2009/093251, incorporated by reference as if fully disclosed herein. The term “derived from” as used herein means that the recombinant polypeptide is a fragment of the representative sigma c protein comprising an amino acid sequence corresponding to amino acid residues 70-326, 117-326 or 122-326 of the sigma C protein of the ARV strain S1133 as set forth in SEQ ID NO: 1. The vaccine may be modified by replacing one polypeptide that is a representative for one group with another polypeptide from the same group.

TABLE 2
Reovirus Isolates (RI) from Israel arranged in four RI groups and SEQ ID NO
(SIN) of sigma C proteins (SCP) arranged in four corresponding SCP groups.

| I | | II | | III | | IV | |
|---|---|---|---|---|---|---|---|
| RI # | SCP SIN² | RI # | SCP SIN² | RI # | SCP SIN² | RI # | SCP SIN² |
| ISR521 | 6 | ISR522 | 12 | ISR524 | 17 | 59103 | 23 |
| ISR526 | 7 | ISR5217 | 13 | ISR527 | 18 | S1133 | 1 |
| ISR5215¹ | 8 | ISR5221 | 14 | ISR528 | 19 | | |
| SR5220 | 9 | ISR5222 | 15 | ISR529 | 20 | | |
| ISR5225 | 10 | ISR5223 | 16 | ISR5211 | 21 | | |
| ISR5226 | 11 | | | ISR5213 | 22 | | |

¹bold: isolates expressing the sigma C protein variant most identical to its group.
²SIN, SEQ ID NO:

For example, one of the at least two different isolated polypeptides may be an isolated polypeptide derived from a sigma c protein that has at least 75% identity to SEQ ID NO: 6-11 (Group I); a polypeptide derived from a sigma c protein that has at least 75% identity to SEQ ID NOs: 12-16 (Group II); a polypeptide derived from a sigma c protein that has at least 75% identity to SEQ ID NOs: 17-22 (Group III); or a polypeptide derived from a sigma c protein that has at least 75% identity to SEQ ID NOs: 1 and 23 (Group IV). The vaccine further includes one, two, three or more additional different polypeptides as defined above. In particular embodiments, in each one of the above relevant combinations, the different polypeptides have an amino acid sequence as set forth in SEQ ID NO: 2, SEQ ID NO: 3, SEQ ID NO: 4 or SEQ ID NO: 5.

In another example, two of the at least two different polypeptides may be a first polypeptide derived from a sigma c protein that has at least 75% identity to SEQ ID NOs: 6-11 (Group I) and a second polypeptide derived from a sigma c protein that has at least 75% identity to SEQ ID NOs: 12-16 (Group II); a first polypeptide derived from a sigma c protein that has at least 75% identity to SEQ ID NOs: 6-11 (Group I) and a second polypeptide derived from a sigma c protein that has at least 75% identity to SEQ ID NOs: 17-22 (Group III); a first polypeptide derived from a sigma c protein that has at least 75% identity to SEQ ID NO: 6-11 (Group I) and a second polypeptide derived from a sigma c protein that has at least 75% identity to SEQ ID NOs: 1 and 23 (Group IV); a first polypeptide derived from a sigma c protein that has at least 75% identity to SEQ ID NO: 12-16 (Group II) and second polypeptide derived from a sigma c protein that has at least 75% identity to SEQ ID NOs: 17-22 (Group III); a first polypeptide derived from a sigma c protein that has at least 75% identity to SEQ ID NOs: 12-16 (Group II) and second polypeptide derived from a sigma c protein that has at least 75% identity to SEQ ID NOs: 1 and 23 (Group IV); or a first polypeptide derived from a sigma c protein that has at least 75% identity to SEQ ID NOs: 17-22 (Group III) and second polypeptide derived from a sigma c protein that has at least 75% identity to SEQ ID NOs: 1 and 23 (Group IV). The vaccine further includes one, two or more additional different polypeptides as defined above.

In particular embodiments, in each one of the above relevant combinations, the two different polypeptides have an amino acid sequence as set forth in SEQ ID NO: 2, SEQ ID NO: 3, SEQ ID NO: 4 or SEQ ID NO: 5.

Alternatively, three of the at least two different polypeptides may be a first polypeptide derived from a sigma c protein that has at least 75% identity to SEQ ID NOs: 6-11 (Group I), a second polypeptide derived from a sigma c protein that has at least 75% identity to SEQ ID NOs: 12-16 (Group II) and a third polypeptide derived from a sigma c protein that has at least 75% identity to SEQ ID NOs: 17-22 (Group III); a first polypeptide derived from a sigma c protein that has at least 75% identity to SEQ ID NOs: 6-11 (Group I), a second polypeptide derived from a sigma c protein that has at least 75% identity to SEQ ID NOs: 12-16 (Group II) and a third polypeptide derived from a sigma c protein that has at least 75% identity to SEQ ID NOs: 1 and 23 (Group IV); a first polypeptide derived from a sigma c protein that has at least 75% identity to SEQ ID NOs: 6-11 (Group I), a second polypeptide derived from a sigma c protein that has at least 75% identity to SEQ ID NOs: 17-22 (Group III) and a third polypeptide derived from a sigma c protein that has at least 75% identity to SEQ ID NOs: 1 and 23 (Group IV); or a first polypeptide derived from a sigma c protein that has at least 75% identity to SEQ ID NOs: 12-16 (Group II), a second polypeptide derived from a sigma c protein that has at least 75% identity to SEQ ID NOs: 17-22 (Group III) and a third polypeptide derived from a sigma c protein that has at least 75% identity to SEQ ID NOs: 1 and 23 (Group IV). In particular embodiments, in each one of the above relevant combinations, the three different polypeptides have an amino acid sequence as set forth in SEQ ID NO: 2, SEQ ID NO: 3, SEQ ID NO: 4 or SEQ ID NO: 5. The vaccine further includes one or more additional different polypeptides as defined above.

In certain embodiments, the vaccine comprises four different polypeptides; or the vaccine comprising mammalian expression vector or the viral vector comprises a nucleic acid molecule encoding four different polypeptides, wherein the first of said four different polypeptides is derived from a sigma c protein that has at least 75% identity to SEQ ID NOs: 6-11 (Group I); the second of said four different polypeptides is derived from a sigma c protein that has at least 75% identity to SEQ ID NOs: 12-16 (Group II); the third of said four different polypeptides is derived from a sigma c protein that has at least 75% identity to SEQ ID NOs: 17-22 (Group III); and the fourth of said four different polypeptides is derived from a sigma c protein that has at least 75% identity to SEQ ID NO: 1 or 23 (Group IV).

In particular embodiments, the first of said four different polypeptides is derived from a sigma c protein that has at least 40%, at least 50%, at least 60%, at least 70%, at least 80%, at least 85%, at least 90%, or at least 95, 96, 97, 98, or 99% identity to SEQ ID NO: 8 (Group I); the second of said four different polypeptides is derived from a sigma c protein that has at least 40%, at least 50%, at least 60%, at least 70%, at least 80%, at least 85%, at least 90%, or at least 95, 96, 97, 98, or 99% identity to SEQ ID NO: 16 (Group II); the third of said four different polypeptides is derived from a sigma c protein that has at least 40%, at least 50%, at least 60%, at least 70%, at least 80%, at least 85%, at least 90%, or at least 95, 96, 97, 98, or 99% identity to SEQ ID NO: 19 (Group III); and the fourth of said four different polypeptides is derived from a sigma c protein that has at least 40%, at least 50%, at least 60%, at least 70%, at least 80%, at least 85%, at least 90%, or at least 95, 96, 97, 98, or 99% identity to SEQ ID NO: 23 (Group IV).

In particular embodiments, the first of said four different polypeptides is derived from a polypeptide selected from the group consisting of SEQ ID NO: 6-11 (Group I); the second of said four different polypeptides is derived from a polypeptide selected from the group consisting of SEQ ID NO: 12-16 (Group II); the third of said four different polypeptides is derived from a polypeptide selected from the group consisting of SEQ ID NO: 17-22 (Group III); and the fourth of said four different polypeptides is derived from a polypeptide selected from the group consisting of SEQ ID NO: 1 and 23 (Group IV). In more particular embodiments, the four different polypeptides have an amino acid sequence as set forth in SEQ ID NO: 2, SEQ ID NO: 3, SEQ ID NO: 4 and SEQ ID NO: 5.

In certain embodiments, any one of the vaccines of the present invention comprises a pharmaceutically acceptable amount of a pharmaceutically acceptable diluent or carrier. Pharmacologically acceptable diluents and carriers suitable for use in the vaccine of the invention may be any conventional liquid carrier suitable for veterinary pharmaceutical compositions, such as a balanced salt solution suitable for use in tissue or cell culture media, e.g. sterile phosphate buffered saline or distilled water. Other suitable media can include emulsions. Non-fat dry milk may be utilized as a carrier in vaccines formulated for application via drinking water.

In certain embodiments, any one of the vaccines of the present invention, whether it comprises a pharmaceutically acceptable amount of a pharmaceutically acceptable diluent or carrier or not, further comprises an adjuvant, such as heat-labile enterotoxin (LT), complete Freund adjuvant, incomplete Freund adjuvant, aluminium hydroxide; and/or a preservative, such as thimerosal or 20% water-in-oil emulsions with for example Marcol 52 mineral oil (ESSO, France).

In yet another aspect, the present invention is directed to any one of the vaccines or viral vectors defined above, for use in vaccination of an avian species or for use in inducing an avian immune response conferring protection against avian reovirus. The vaccine or viral vector may be used along with a pharmaceutically acceptable amount of a pharmaceutically acceptable diluent or carrier and/or an adjuvant as described above.

In yet an additional aspect, the present invention is directed to a method for vaccinating an avian species or for inducing an avian immune response conferring protection against avian reovirus, which comprises administering any one of the vaccines or viral vectors defined above to a bird. In particular embodiments, the vaccine comprises a combination of four isolated polypeptides as described above. The vaccine or viral vector may be administered along with a pharmaceutically acceptable amount of a pharmaceutically acceptable diluent or carrier and/or an adjuvant as described above.

In certain embodiments, the avian species that can be vaccinated using any one of the vaccines and methods described herein is any bird species which is produced commercially, such as poultry, e.g. chickens, turkeys, ducks, geese, pheasants, pigeons or guinea fowl.

In certain embodiments, the use or method disclosed above induces the production of significantly higher, for example statistically significantly higher, systemic levels of neutralizing anti-sigma C protein antibody as compared with the systemic levels of said antibody obtained after administration to said bird of full length sigma C protein of the ARV strain S1133.

In certain embodiments the vaccine may be administered by any suitable route of administration such as by injection, intradermally or subcutaneously; or orally via the drinking water.

The invention will now be illustrated by the following non-limiting examples.

### EXAMPLES

### Materials and Methods

Expression of the SC protein

Three cDNA fragments of SC from the vaccine strain s1133 encoding SC residues 1-326, 122-326 and 192-326 were produced by polymerase chain reaction (PCR) with specifically designed oligonucleotides (Table 3). Fragments 122-326 and 192-326 were cleaved with restriction enzymes BamHI and Eagl introduced into the primers during synthesis, and were

TABLE 3
Designed oligonucleotides for amplification of sigma C (SC) fragments.

| | | | Expected size |
|---|---|---|---|
| Labelª | Direction | Primer sequences (5′→3′) | (bp) |
| SC122-326 | Forward | GGGGGATCCGACGGAAACTCCACTGCC | 669 |
| SC122-326 | Reverse | GGGCGGCCGTTAGGTGTCGATGCCGG | |
| SC192-326 | Forward | GGGGGATCCTCGGCGGAGGCTCAACTAATGC | 459 |
| SC192-326 | Reverse | GGGCGGCCGTTAGGTGTCGATGCCGG | |

ªOligonucleotides were designed to amplify the gene encoding SC122-326 and SC192-326 partial proteins; the 5′ends of the oligonucleotides were designed to create restriction enzyme sites BamHI-EagI, BamHI-EagI, and EcoRI-EcoRI (underlined), respectively, after PCR amplification.

cloned into the expression vector pET28a (Novagen, Darmstadt, Germany), which included a purification tag containing six consecutive histidine residues at the N terminus. The sequence of the insert was confirmed by DNA-sequence analysis (Hy-Labs, Rehovot, Israel). For expression, Escherichia coli strain BL21 (DE3) was freshly transformed with the plasmid. Cultures were grown aerobically at 37° C. to an optical density (OD) at 600 nm of 0.6-0.8. The cultures were cooled to below 25° C., and expression was induced by adding 1 mM isopropyl β-D-1-thiogalactopyranoside (IPTG) and incubating for 3 h at 25° C. Harvested cells were resuspended in 40 ml cold resuspension buffer (4.29 mM Na₂HPO₄, 1.47 mM KH₂PO₄, 2.7 mM KCl, 137 mM NaCl, 0.1% v/v Tween-20) and frozen at −20° C. (Guardado-Calvo et al. 2009). Bacteria were lysed by sonicating three times for 10 min each time (Sonics, Taunton, MA, USA), centrifuged (10,000×g for 15 min at 4° C.) and the pellet was discarded. SC residue 1-326 was expressed as described previously (Goldenberg et al. 2011). The expressed SC122-326 protein fragment was purified in a Ni-NTA agarose column according to the manufacturer's instructions (Qiagen, Hilden, Germany). The purified protein was dialyzed overnight against phosphate buffered saline (PBS) at 4° C. The expressed SC proteins were detected by 12% SDS-polyacrylamide gel electrophoresis (PAGE). The amount of SC protein was estimated by comparison with a standard curve of known amounts of bovine serum albumin run on the same gel.
Cloning and expression of SC in Pichia pastoris

A DNA fragment encoding SC residues 1-326 was produced by PCR with specifically designed oligonucleotides (Table 3). This fragment was cleaved by the restriction enzyme EcoRI (GAATTC), which was introduced by the primers during synthesis. The isolated fragment was ligated to plasmid pHILD2 provided in the P. pastoris kit (Invitrogen, San Diego, CA, USA) as described previously (Pitcovski et al. 2003). Briefly, the ligated DNA construct was transformed into E. coli XL1-blue cells and white colonies that grew on LB (luria-bertani) plates containing ampicillin (100 g/ml), X-Gal to final concentration of 80 μg/ml and IPTG to final concentration of 20 mM were isolated. Following characterization, a plasmid harboring SC with the correct sequence was cloned into P. pastoris as follows.

The vector was linearized and introduced into P. pastoris cells, resulting in the insertion of the transgene at the AOX1 locus. Positive colonies were grown and recombinant protein production was induced by adding methanol. Following induction, cells were lysed by vortexing with glass beads, centrifuged, and the supernatant containing the soluble SC was analyzed by SDS-PAGE.

### Enzyme-Linked Immunosorbent Assay (ELISA)

To determine the anti-SC antibody titer, SC was used as the antigen in an ELISA. The antigen was diluted in coating buffer (0.397 g Na₂CO₃, 0.732 g NaHCO₃, 250 ml double-distilled water pH 9.6) and incubated in an ELISA plate (Nunc, Rochester, NY, USA) for 24 h at 4° C. Each subsequent step was followed by three washes with 0.05% Tween-20 in PBS. Serum from birds vaccinated with the tested proteins or controls were serially double-diluted (1:100-1:800,000) in a blocking buffer (5% w/v skim milk, 0.05% Tween-20 in PBS) and incubated for 1 h. The secondary antibody, peroxidase-conjugated rabbit anti-chicken IgG (Sigma, Rehovot, Israel), diluted 1:7000, was added and the mixture was incubated for 1 h. The substrate o-phenylenediamine dihydrochloride (Sigma) was then added. OD was measured by ELISA reader (Thermo Scientific Multiskan RC, Vantaa, Finland) at 450 nm. The endpoint titer was determined as the last dilution for which the OD was still positive (relative to the negative control in the ELISA).

The level of anti-ARV antibodies in the sera of vaccinated birds was determined by a commercial ELISA (IDEXX® Laboratories, USA) according to the manufacturer's instructions. OD values were measured at 650 nm. Sample-to-positive (S/P) ratios greater than 0.2 were considered to be positive for ARV (S/P ratio=sample mean OD)−negative control mean OD/positive control mean OD−negative control mean OD).

### Cell-Proliferation Assay

Spleens were collected from birds 42 days post-vaccination and macerated with a syringe plunger through a screen sieve to obtain a single-cell suspension in PBS. Splenocytes were suspended in RPMI 1640 supplemented with 2% fetal bovine serum, 2 mM L-glutamine, penicillin (100 U/ml) and streptomycin (10 ng/ml) (Biotech Industry, Bet Haemek, Isarel). Cells (1×10⁶ per well) were seeded in 96-well culture plates. Concanavalin A (ConA; 5 pg/m1), lipopolysaccharide (LPS; 5 μg/ml) or ARV (5 μl/well, at a titer of 10^{6.6}) (Sigma Aldrich) were added as stimulators in triplicate and incubated for 48 h. Cell titer blue (CTB) assay was performed by adding 20 μl CTB reagent (Promega, Madison, WI, USA) to each well. The cells were then incubated for 6 h at 37° C. under 5% CO₂. Color intensity of the CTB reagent was quantified by fluorometer at excitation/emission wavelengths of 560 and 590 nm, respectively. The measured absorbance was proportional to the number of viable cells. Results were calculated and presented as stimulation index (SI), representing the ratio between stimulated cells and non-stimulated cells.

Neutralization Test of Virulent ARV Strain s1133

The ability of the sera to neutralize virulent reovirus was tested by monitoring inhibition of embryonic mortality 3-7 days post-inoculation. The tested sera were filtered and heated for 30 min at 56° C. to inactivate complement activity. The virus was diluted with PBS in a series of 10-fold dilutions. Mixtures of equal volumes of diluted virus and sera (or PBS as a negative control) were incubated for 40 min at room temperature. Each of the mixtures was inoculated into five fertile 7-day-old SPF embryonated eggs.

To confirm the virulence potential of the indicator virus, 10 eggs were inoculated with the undiluted virus that was mixed 1:1 with PBS. In addition, in several of the experiments, virus-free sera were inoculated into embryonated eggs to confirm the absence of nonspecific mortality due to serum constituents. All eggs were illuminated daily in a dark room to determine embryo viability, and the number of live and dead embryos was recorded.

The neutralization index (NI) was determined as the ratio between the highest virus dilution that killed at least 95% (cumulative) of the embryos and the virus dilution that killed the same percentage of embryos in the presence of antibodies. NI value was expressed as the ratio between the log of the dilutions. A NI of 2 or greater in the antiserum was considered as having neutralized the virus.

TABLE 4
Vaccination program.

| Group | Vaccine at 14 d^{a} | Vaccine at 28 d^{a} |
|---|---|---|
| 1 | ARV s1133^{b} | ARV s1133 |
| 2 | PBS + adjuvant^{c} | PBS + adjuvant |
| 3 | SC1-326 | SC1-326 |
| 4 | SC122-326 | SC122-326 |
| 5 | SC192-326 | SC192-326 |
| 6 | SC122-326 | ARV s1133 |

^{a}Days of age;
^{b}Positive control;
^{c}Negative control.

### Statistics

ELISA, neutralization and cell-proliferation results were analyzed by one-way analysis of variance (ANOVA) with Tukey test. All of the analyses were performed with GraphPadPrism5 software.

### Example 1. SC Protein Expression

Following analysis of the SC protein structure, cDNA fragments encoding three polypeptides residues: 1-326, 122-326, 192-326, were produced, cloned, and expressed in E. coli. A polyhistidine tag was added at the 5′ end to allow for detection and purification of the expressed proteins. The resultant proteins were partially purified. The expressed SC fragments were at expected sizes of 36 kD, 23 kD and 15kD (respectively) as determined on a 12% SDS-polyacrylamide gel.

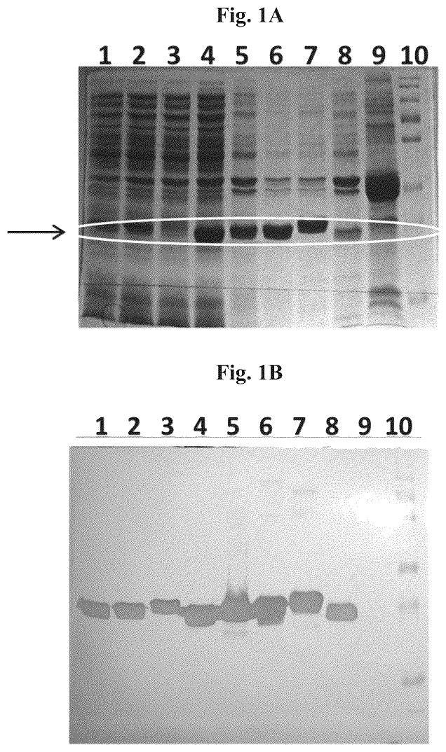

FIG. 1 shows the efficient expression of the 122-326 fragment of four different reo virus isolates: SC1133, SC528, SC5223 and SC5215.

### Example 2. Antibody Response to Vaccination

Following vaccination, the immunogenicity of the SC proteins and the ability of the produced antibodies to detect the virus were tested by ELISA. Using SC122-326 as the antigen, antibodies derived following vaccination with proteins SC122-326 and SC192-326 detected the antigen at high titers of 1:800,000. The mean of the titers of birds in this group were significantly higher (P<0.001) than those in the negative control, whereas the mean of the antibody titer derived following vaccination with SC1-326 protein were similar to those of the negative control (FIG. 2).

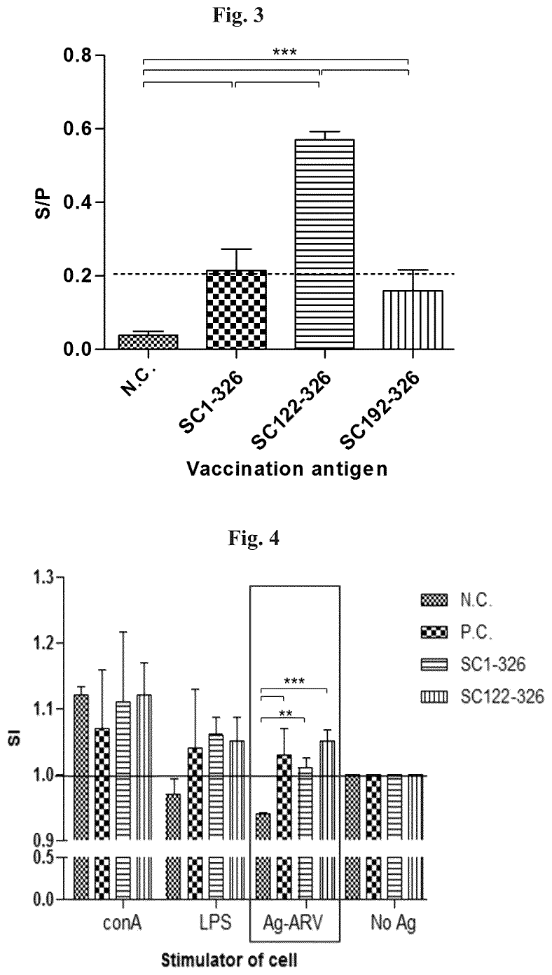

Antibody level against whole virus following vaccination with SC192-326 or the negative control reached a value of 0.2 or lower, whereas antibodies derived following vaccination with SC1-326 or SC122-326 were positive (0.25 and 0.5, respectively) (FIG. 3). The S/P in SC122-326 group was significantly higher than in the other groups.

Similarly, following vaccination, the immunogenicity of the SC122-326 fragments of the four different RI Groups (SEQ ID NOs: 2, 3, 4 and 5) and the ability of the produced antibodies to detect the virus are tested by ELISA.

### Example 3. Cell Proliferation

Splenocytes from the vaccinated chickens were examined for antigen-specific cell proliferation. Cells were treated with ConA as a control for proliferation of T cells, LPS as a control for proliferation of B cells (positive controls), PBS (negative control) and ARV (specific antigen). In the CTB assay, the measured absorbance was proportional to the number of viable cells. Results are presented as SI (mitogen- or antigen-stimulated/nonstimulated cells).

The positive control treatments exhibited proliferation following the stimulus. Cells stimulated with ARV showed significant differences among treatment groups. Cells from the group immunized with the virus or SC122-326 showed significant proliferation as compared to the negative control (P<0.01), whereas proliferation in group that were injected with SC1-326 was significantly lower than SC122-326 or the positive control groups (P<0.05) (FIG. 4).

Similarly, splenocytes from chickens vaccinated with a combination vaccine comprising polypeptides of SEQ ID NOs: 2, 3, 4 and 5 are examined for antigen-specific cell proliferation.

### Example 4. Effect of SC122-326 in Priming the Response Against ARV

The effect of vaccination with SC122-326 as compared to vaccination with the whole-virus vaccine as an inducer of a primary response against ARV was tested. One group of chickens was injected with the ARV strain s1133 vaccine and the other with SC122-326. Two weeks later, ARV s1133 was injected into both groups. Primary vaccination with ARV or SC122-326, induce a similar significant elevation following secondary vaccination with whole ARV (FIG. 5).

Similarly, the effect of vaccination with a combination vaccine comprising polypeptides of SEQ ID NOs: 2, 3, 4 and 5 as compared to vaccination with the whole-virus vaccine as an inducer of a primary response against ARV is tested.

### Example 5. Virus Neutralization

Sera from vaccinated birds were tested for neutralization ability against the virulent virus in SPF embryonated eggs. No protection was conferred by sera produced by the negative control birds or from the group injected with the SC1-326 (NI=0). In contrast, serum produced by birds injected with the protein SC122-326 conferred protection (neutralization capacity) similar to that achieved with antibodies that developed following vaccination with whole virus (NI>4) (Table 3).

Similarly, sera from birds vaccinated with a combination vaccine comprising polypeptides of SEQ ID NOs: 2, 3, 4 and 5 are tested for neutralization ability against the virulent virus in SPF embryonated eggs.

TABLE 5
Virus neutralization by antibodies
against ARV or SC fragments.

| Vaccination | Neutralization index^{a} |
|---|---|
| Adjuvant only | 0 |
| (negative control) | |
| ARV s1133 | 4 |
| SC1-326 | 0 |
| SC122-326 | >4 |

^{a}Neutralization index (NI) value of 2 or higher is considered as neutralizing against that virus.

### References

Benavente, J. & Martínez-Costas, J., 2007. Avian reovirus: Structure and biology. Virus Research, 123(2), pp.105-119. Available at: http://linkinghub.elsevier.com/retrieve/pii/S0168170206002772.
Fingerut, E. et al., 2003. A subunit vaccine against the adenovirus egg-drop syndrome using part of its fiber protein. Vaccine, 21, pp.2761-2766.
Goldenberg, D. et al., 2011. Differentiating infected from vaccinated animals, and among virulent prototypes of reovirus. Journal of Virological Methods, 177(1), pp.80-86. Available at: http://dx.doi.org/10.1016/j.jviromet.2011.06.023.
Goldenberg, D. et al., 2010. Genetic and antigenic characterization of sigma C protein from avian reovirus. Avian pathology : journal of the W.V.P.A, 39(3), pp.189-199.
Grande, A. N. A. et al., 1997. Protein Architecture of Avian Reovirus S1133 and Identification of the Cell Attachment Protein., 71(1), pp.59-64.
Guardado Calvo, P. et al., 2005. Structure of the Carboxy-terminal Receptor-binding Domain of Avian Reovirus Fibre SigmaC. Journal of Molecular Biology, 354(1), pp.137-149. Available at: http://linkinghub.elsevier.com/retrieve/pii/S0022283605010995.
Guardado-Calvo, P. et al., 2009. Crystallographic structure of the α-helical triple coiled-coil domain of avian reovirus S1133 fibre. Journal of General Virology, 90(2009), pp.672-677.
van der Heide, L., Kalbac, M. & Brustolon, M., 1983. Development of an attenuated apathogenic reovirus vaccine against viral arthritis/tenosynovitis. Avian diseases, 27(3), pp.698-706. Available at: http://www.ncbi.nlm.nih.gov/pubmed/6314976 [Accessed Dec. 9, 2015].
Jones, R. C., 2000. Avian reovirus infections. Revue scientifique et technique (International Office of Epizootics), 19(2), pp.614-25. Available at: http://www.ncbi.nlm.nih.gov/pubmed/10935283 [Accessed Dec. 9, 2015].
Jung, K. M. et al., 2014. Use of IgY antibody to recombinant avian reovirus σC protein in the virus diagnostics. Acta virologica, 58(2), pp.108-13. Available at: http://www.ncbi.nlm.nih.gov/pubmed/24957714 [Accessed Dec. 9, 2015].
Kant, A. et al., 2003. Classification of Dutch and German avian reoviruses by sequencing the sigma C protein. Veterinary research, 34(2), pp.203-12. Available at: http://www.ncbi.nlm.nih.gov/pubmed/12657212 [Accessed Dec. 9, 2015].
Lu, H. et al., 2015. Isolation and molecular characterization of newly emerging avian reovirus variants and novel strains in Pennsylvania, USA, 2011-2014. Scientific Reports, 5(April), p.14727. Available at: http://www.nature.com/articles/srep14727.
Lublin, A. et al., 2011. Wide-range protection against avian reovirus conferred by vaccination with representatives of four defined genotypes. Vaccine, 29(47), pp.8683-8688. Available at: http://dx.doi.org/10.1016/j.vaccine.2011.08.114.
McAleer, W. J. et al., 1984. Human hepatitis B vaccine from recombinant yeast. Nature, 307(5947), pp.178-80. Available at: http://www.ncbi.nlm.nih.gov/pubmed/6318124 [Accessed Dec. 9, 2015].
Pitcovski, J. et al., 2005. A subunit vaccine against hemorrhagic enteritis adenovirus. Vaccine, 23, pp.4697-4702.
Pitcovski, J. et al., 2003. Development and large-scale use of recombinant VP2 vaccine for the prevention of infectious bursal disease of chickens. Vaccine, 21, pp.4736-4743.
Shapouri, M. R. et al., 1995. Cloning, sequencing and expression of the S1 gene of avian reovirus. The Journal of general virology, 76 (Pt 6)(1995), pp.1515-20. Available at: http://www.ncbi.nlm.nih.gov/pubmed/7782781.
Shapouri, M. R. S. et al., 1996. Characterization of monoclonal antibodies against avian reovirus strain S1133. Avian pathology: journal of the W.V.P.A, 25(1), pp.57-67. Available at: http://www.ncbi.nlm.nih.gov/pubmed/18645837 [Accessed Dec. 9, 2015].
Troxler, S. et al., 2013. Identification of a new reovirus causing substantial losses in broiler production in France, despite routine vaccination of breeders. The Veterinary record, 172(21), p.556. Available at: http://www.ncbi.nlm.nih.gov/pubmed/23636701 [Accessed Dec. 9, 2015].
Valentino, K. & Poronsky, C. B., 2015. Human Papillomavirus Infection and Vaccination. Journal of pediatric nursing. Available at: http://www.sciencedirect.com/science/article/pii/S0882596315003267 [Accessed Nov. 22, 2015].
Vasserman, Y. et al., 2004. The influence of reovirus sigma C protein diversity on vaccination efficiency. Avian diseases, 48(2), pp.271-8. Available at: http://www.ncbi.nlm.nih.gov/pubmed/15283414 [Accessed Dec. 9, 2015].
Wu, H. et al., 2005. Yeast-derived sigma C protein-induced immunity against avian reovirus. Avian diseases, 49(2), pp.281-4. Available at: http://www.ncbi.nlm.nih.gov/pubmed/16094835 [Accessed Dec. 9, 2015].

1. An isolated polypeptide comprising an amino acid sequence corresponding to the amino acid residues forming a full or partial α-helical domain, the hinge domain, the β-triple spiral domain and a full or partial globular head domain of an avian reovirus sigma C protein, and lacking the amino acid sequence that is N-terminal to said α-helical domain.

2. The isolated polypeptide of claim 1, comprising an internal amino acid sequence corresponding to amino acid residues 70-326, 117-326 or 122-326 of the sigma C protein of the ARV strain S1133 as set forth in SEQ ID NO: 1.

3. The isolated polypeptide of claim 2, wherein said internal amino acid sequence has at least 40%, at least 50%, at least 60%, at least 70%, at least 80%, at least 85%, at least 90%, or at least 95, 96, 97, 98, or 99% identity to amino acid residues 70-326, 117-326 or 122-326 of the sigma C protein of the ARV strain S1133 as set forth in SEQ ID NO: 1.

4. The isolated polypeptide of any one of claims 1 to 3 that blocks or reduces the binding of infectious avian reovirus to the native receptor of sigma C protein of an avian reovirus.

5. The isolated polypeptide of any one of claims 1 to 4, which, when administered to a bird, optionally in combination with an adjuvant, induces a protective immune response against an infectious avian reovirus.

6. The isolated polypeptide of claim 5, which, when administered to a bird, induces the production of significantly higher systemic levels of neutralizing anti-sigma C protein antibody as compared with the systemic levels of said antibody obtained after administration to a bird of full length sigma C protein of the ARV strain S1133.

7. The isolated polypeptide of claim 3 comprising an amino acid sequence having at least 40%, at least 50%, at least 60%, at least 70%, at least 80%, at least 85%, at least 90%, or at least 95, 96, 97, 98, or 99% identity to an internal amino acid sequence of a sigma C protein selected from the group disclosed in Table 1, said internal amino acid sequence corresponding to amino acid residues 70-326, 117-326 or 122-326 of the sigma C protein of the ARV strain S1133 as set forth in SEQ ID NO: 1.

8. The isolated polypeptide of claim 7 comprising an amino acid sequence within an internal amino acid sequence of a sigma C protein selected from the group disclosed in Table 3, said internal amino acid sequence corresponds to amino acid residues 70-326, 117-326 or 122-326 of the sigma C protein of the ARV strain S1133 as set forth in SEQ ID NO: 1.

9. The isolated polypeptide of claim 8 comprising an amino acid sequence selected from the group consisting of SEQ ID NO: 2, SEQ ID NO: 3, SEQ ID NO: 4 and SEQ ID NO: 5.

10. The isolated polypeptide of any one of claims 1 to 9, further comprising a tag for identification and/or purification, such as a polyhistidine tag.

11. A nucleic acid molecule comprising a nucleic acid sequence encoding at least one isolated polypeptide of any one of claims 1 to 10.

12. An expression vector comprising a control element, such as a promoter, operably linked to said nucleic acid molecule of claim 11, wherein said expression vector is designed to replicate and express relevant genes in for example a bacterial, yeast or insect cell.

13. A vaccine comprising at least one isolated polypeptide of any one of claims 1 to 10.

14. The vaccine of claim 13 further comprising an adjuvant, such as heat-labile enterotoxin (LT), complete Freund adjuvant, incomplete Freund adjuvant, aluminium hydroxide; and/or a preservative, such as thimerosal or 20% water-in-oil emulsions with for example Marcol 52 mineral oil (ESSO, France).

15. A vaccine comprising a mammalian expression vector, such as pcDNA3, comprising a control element, such as a promoter, operably linked to said nucleic acid molecule of claim 11.

16. A viral vector, such as a recombinant Marek's disease (MD) virus, comprising a control element, such as a promoter, operably linked to said nucleic acid molecule of claim 11.

17. The vaccine of claim 13 or 14 comprising at least two different polypeptides; or the vaccine of claim 15 or the viral vector of claim 16, wherein said nucleic acid molecule encodes at least two different polypeptides; each one of said at least two different polypeptides is derived from a representative of one of two, three or four groups of different sigma c proteins, wherein the defining feature of each group is that the amino acid sequences of the different sigma c proteins of said group has at least 75% identity.

18. The vaccine of any one of claims 13 to 15 and 17 comprising four different polypeptides; or the viral vector of claim 16 or 17 comprising a nucleic acid molecule encoding four different polypeptides, wherein the first of said four different polypeptides is derived from a sigma c protein that has at least 75% identity to SEQ ID NOs: 6-11 (Group I); the second of said four different polypeptides is derived from a sigma c protein that has at least 75% identity to SEQ ID NOs: 12-16 (Group II); the third of said four different polypeptides is derived from a sigma c protein that has at least 75% identity to SEQ ID NOs: 17-22 (Group III); and the fourth of said four different polypeptides is derived from a sigma c protein that has at least 75% identity to SEQ ID NOs: 1 and 23(Group IV).

19. The vaccine or the viral vector of claim 18, wherein the first of said four different polypeptides is derived from a sigma c protein that has at least 40%, at least 50%, at least 60%, at least 70%, at least 80%, at least 85%, at least 90%, or at least 95, 96, 97, 98, or 99% identity to SEQ ID NO: 8; the second of said four different polypeptides is derived from a sigma c protein that has at least 40%, at least 50%, at least 60%, at least 70%, at least 80%, at least 85%, at least 90%, or at least 95, 96, 97, 98, or 99% identity to SEQ ID NO: 16; the third of said four different polypeptides is derived from a sigma c protein that has at least 40%, at least 50%, at least 60%, at least 70%, at least 80%, at least 85%, at least 90%, or at least 95, 96, 97, 98, or 99% identity to SEQ ID NO: 19; and the fourth of said four different polypeptides is derived from a sigma c protein that has at least 40%, at least 50%, at least 60%, at least 70%, at least 80%, at least 85%, at least 90%, or at least 95, 96, 97, 98, or 99% identity to SEQ ID NO: 23.

20. The vaccine or the viral vector of claim 18, wherein the first of said four different polypeptides is derived from a polypeptide selected from the group consisting of SEQ ID NO: 6-11 (Group I); the second of said four different polypeptides is derived from a polypeptide selected from the group consisting of SEQ ID NO: 12-16 (Group II); the third of said four different polypeptides is derived from a polypeptide selected from the group consisting of SEQ ID NO: 17-22 (Group III); and the fourth of said four different polypeptides is derived from a polypeptide selected from the group consisting of SEQ ID NO: 1 and 23 (Group IV).

21. The vaccine of claim 20, wherein said four different polypeptides have an amino acid sequence as set forth in SEQ ID NO: 2, SEQ ID NO: 3, SEQ ID NO: 4 and SEQ ID NO: 5.

22. The vaccine of any one of claims 13 to 15 and 17 to 21, or the viral vector of any one of claims 16 to 21, for use in vaccination of an avian species against avian reovirus or for inducing an avian immune response conferring protection against avian reovirus.

23. A method for vaccination of an avian species against avian reovirus or for inducing an avian immune response conferring protection against avian reovirus, which comprises administering a vaccine of any one of claims 13 to 15 and 17 to 21 or the viral vector of any one of claims 16 to 21 to a bird.

24. The method of claim 23, which induces the production of significantly higher systemic levels of neutralizing anti-sigma C protein antibody as compared with the systemic levels of said antibody obtained after administration to said bird of full length sigma C protein of the ARV strain S1133.

25. The method of claim 23 or 24, comprising administering said vaccine to birds by injection, intradermally or subcutaneously; or orally via the drinking water.
